(19) United States
(12) Patent Application Publication
(10) Pub. No.: US 2014/0329756 A1
(43) Pub. Date: Nov. 6, 2014

(54) TEMPERATURE-RELEASE CATALYST FOR CROSS-LINKING HALYURONIC ACID DURING INJECTION

(71) Applicants: Phi Nguyen, Houston, TX (US); Loc Phan, San Jose, CA (US); Bao Tran, Saratoga, CA (US); Thuan Nguyen, Houston, TX (US)

(72) Inventors: Phi Nguyen, Houston, TX (US); Loc Phan, San Jose, CA (US); Bao Tran, Saratoga, CA (US); Thuan Nguyen, Houston, TX (US)

(73) Assignee: MIBA MEDICAL INC., San Jose, CA (US)

(21) Appl. No.: 14/255,963

(22) Filed: Apr. 18, 2014

Related U.S. Application Data
(60) Provisional application No. 61/722,221, filed on Nov. 4, 2012.

(51) Int. Cl. A61K 31/728; A61K 45/06

(52) U.S. Cl. CPC A61K 31/728; A61K 45/06
USPC 514/17.2; 514/54; 514/57; 514/21.2; 514/23

(57) ABSTRACT

Systems and methods for cosmetic augmentation by forming a biocompatible cross-linked polymer having a multi-phase mixture with a temperature activated catalyst; injecting the mixture into a patient as a homogeneous fluid; activating the catalyst to cross-link the polymer at a predetermined temperature in a patient; and augmenting soft tissue with the biocompatible cross-linked polymer.

This application claims priority to Provisional Application Ser. 61/722,221 filed Nov. 4, 2012, and PCT Application Serial PCT/VN2013/000001 filed Apr. 12 2013, PCT/VN2013/000002 filed Apr. 12, 2013, PCT/VN2013/000003 filed on Apr. 12, 2013, PCT/VN2013/000004 filed Apr. 12, 2013, and PCT/VN2012/000008 filed Dec. 17, 2012, the contents of which are incorporated by reference.

### BACKGROUND

The present invention relates to biodegradable hyaluronic acid (HA) filler compositions for soft tissue implants such as dermal fillers or breast or butt or body implants.

Hyaluronic acid is a substance that is naturally present in the human body. It is found in the highest concentrations in fluids in the eyes and joints. The hyaluronic acid that is used as medicine is extracted. People take hyaluronic acid for various joint disorders, including osteoarthritis. It can be taken by mouth or injected into the affected joint by a healthcare professional. The FDA has approved the use of hyaluronic acid during certain eye surgeries including cataract removal, corneal transplantation, and repair of a detached retina and other eye injuries. It is injected into the eye during the procedure to help replace natural fluids. Young, healthy-looking skin contains an abundance of HA as a naturally hydrating substance. HA is also used as a lip filler in plastic surgery. Some people apply hyaluronic acid to the skin for healing wounds, burns, skin ulcers, and as a moisturizer. Hyaluronic acid works by acting as a cushion and lubricant in the joints and other tissues. In addition, it might affect the way the body responds to injury.

As people age, sunlight and other factors can reduce the amount of HA in their skin. The lack of HA causes skin to lose structure and volume, creating unwanted facial wrinkles and folds—like those parentheses lines around the nose and mouth. Injectable dermal implants are a popular solution to a wide variety of facial contour defects from lip augmentation to plumping up depressed scars. Often used as tissue replacement for victims of serious accidents, injectable dermal implants are very effective in cosmetic surgery procedures such as lip augmentation and scar removal. Using a dermal filler is a safe and effective way to replace the HA the skin has lost, bringing back its volume and smoothing away facial wrinkles and folds.

In a parallel trend, millions of women have undergone breast or butt or body augmentation and reconstruction in the past few decades. Most women choose augmentation to enhance the size and shape of one or both breast or butt or other body parts for personal or aesthetic reasons. In contrast, women who undergo a reconstruction procedure want to reconstruct a breast or butt or body portions that has been removed, typically for health reasons, such as tumor removal. Conventional implants for treating breast or butt or body augmentation or reconstruction include a shell or envelope that is filled with a filler composition, for example, silicone gel, saline solution, or other suitable filler. It is desirable that the filler have lubricating properties to prevent shell abrasion, remain stable over long periods of time, be non-carcinogenic and non-toxic, and have physical properties to prevent skin wrinkling, capsular contracture formation, and implant palpability.

In response to the failures of saline and silicone-gel implants, there have been a number of attempts to make a prosthesis filled with a non-toxic filler that that mimics the shape and feel of a natural breast or butt or body provided by silicone-gel yet is safe to the immune system like saline. Other attempts to provide a safe filler material include polyethylene glycol. However, the triglyceride oil or honey fails to provide an implant that is aesthetically pleasing and also duplicates the touch and feel of a natural breast or butt or body due to the low viscosity of the fillers. Due to the limited options and the inadequacy of current fillers to achieve the desired results, there is a need for safe and efficacious fillers.

### SUMMARY

In one aspect, systems and methods for cosmetic augmentation by forming a biocompatible cross-linked polymer having a multi-phase mixture with a temperature activated catalyst; injecting the mixture into a patient as a homogeneous fluid; activating the catalyst to cross-link the polymer at a predetermined temperature in a patient; and augmenting soft tissue with the biocompatible cross-linked polymer.

In another aspect, systems and methods are disclosed for breast or butt or body implants by forming a biocompatible cross-linked polymer having a multi-phase mixture with a predetermined controlled release of selected pharmaceutical substance to modulate soft tissue response to the polymer; injecting the mixture into a patient and releasing a catalyst at a predetermined body temperature to cause cross-linking the polymer in the patient; filling a semi-permeable shell with the pharmaceutical substance; and augmenting soft tissue with the biocompatible cross-linked polymer.

Advantages of the system may include one or more of the following. The flow properties are tailored for injection through a small bore needle. The system has greater flexibility to control physical properties of the final gel. The final gel could be tailored to have greater cohesive strength which will resist migration to another anatomical space. The final gel durometer could be tailored to be more natural and tissue-like. The final gel could be tailored to have properties similar to surround tissue. The longevity of the final gel could be tailored to meet various anatomical location requirements (longer biodegradation or shorter depending on anatomical location). The final gel physical properties stay constant over the life time of the material.

### BRIEF DESCRIPTION OF THE DRAWINGS

FIG. 1 shows an exemplary block diagram of a computer controlled hyaluronic acid (HA) injector system that cross-links the HA for a predetermined period after a time release catalyst is injected into the body.

FIGS. 2-3 shows an exemplary manual hyaluronic acid (HA) injector system that cross-links the HA for a predetermined period after a time release catalyst is injected into the body.

FIG. 4 shows an exemplary breast or butt or body implant delivery system.

FIG. 5 shows an exemplary process to inject and cross-link materials using temperature-released catalyst.

FIG. 6 shows another exemplary process to inject and cross-link materials using temperature-released catalyst.

### DESCRIPTION

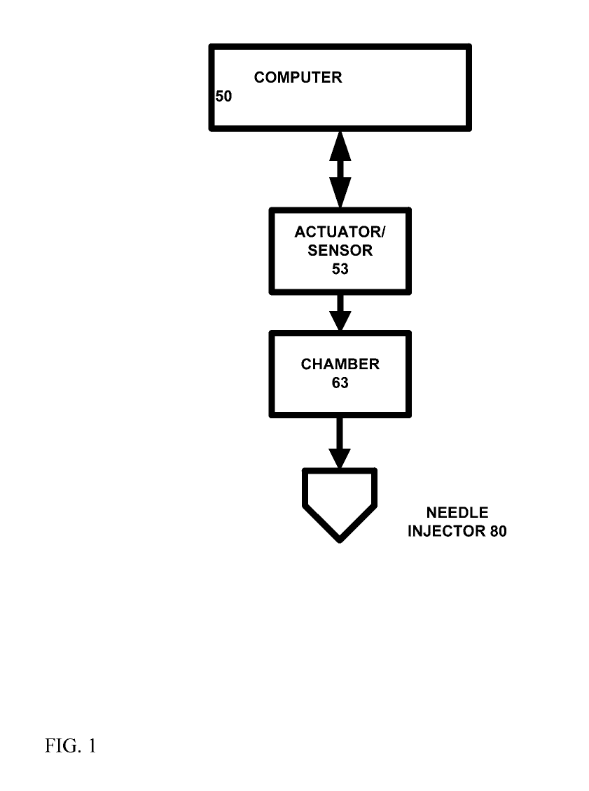

FIG. 1 shows an exemplary block diagram of a hyaluronic acid (HA) injector system that cross-links the HA while the drug is injected into the body. As shown therein, a cartridge with a container 63 housing a flowable component to be mixed when desired in a static mixer. The container 63 terminates in an outlet tip from which the components mixed by the static mixer are expelled. The static mixer may be separable from and attached to the containers or chambers in a manner known per se. The containers, usually made of a plastics material, are joined by a bridge defining an outlet in which the two components are separated by an internal dividing wall to maintain the components separate and unmixed until they reach inlet of the static mixer for mixing therein. In a conventional manner, the static mixer, again usually of a plastics material, comprises a static mixer element housed in an elongate member extending from attachment to the outlet to outlet tip. Also the static mixer element comprises an axially extending serial plurality of alternating oppositely oriented helically twisted mixer blades which act in concert to efficiently and thoroughly mix the separate components as they flow through the static mixer 6 from the outlet to the outlet tip. Pistons or motorized actuators are operated simultaneously by a suitable mechanism (not shown) with the cartridge being retained by the back plate, to dispense the components simultaneously from the containers through the outlet and static mixer to the outlet tip. In this embodiment, the actuators are controlled by a computer for precise mixing and delivery as desired. In one embodiment, a plurality of outlets can be provided so that a plurality of patient areas can be injected in parallel.

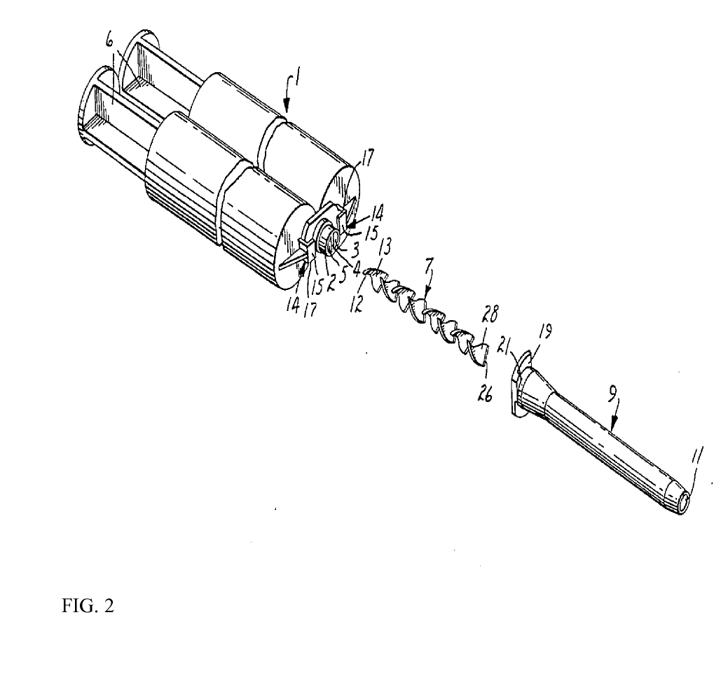

In the embodiment of FIG. 1, the HA can be stored in one chamber and a temperature release catalyst and a crosslinking material can be in the same chamber. In the embodiment of FIG. 2, the HA, temperature release catalyst, and crosslinking material are stored in different chambers. The mixing of the HA, catalyst and crosslinkers can be done outside and at a high viscosity for ease of injection. In either FIG. 1 or FIG. 2, the use of temperature activated catalyst allows the HA to be injected into the body with high viscosity for ease of delivery and once inside the body, the catalyst is activated by temperature to cause cross-linking of the HA to occur and result in a strong dermal implant that retains shape and migrate slowly.

In one embodiment, a gel cap that dissolves at a predetermined body temperature exposes the catalyst to the HA and crosslinkers to cause crosslinking to occur. The catalyst can be biodegradable microencapsulated microspheres or microbeads whose shells dissolve at the predetermined temperature to expose the catalyst for crosslinking in one embodiment.

In another embodiment, a temperature degradable microsphere can be used. The process for preparing biodegradable temperature dissolving microspheres containing the catalyst include the following steps: i) preparing a polymer solution containing the catalyst by dissolving a biodegradable polymer in a water soluble organic solvent followed by dissolving or suspending the physiologically active agent in the polymer solution; ii) forming an O/O emulsion by emulsifying the biological agent containing polymer solution into a water soluble alcohol which contains an emulsion stabilizer; iii) extracting the water soluble organic solvent and water soluble alcohol by adding the O/O emulsion into a neutral or alkaline aqueous solution, and precipitating and reducing the microspheres containing the catalyst.

In one embodiment, the biodegradable polymer of the present invention is a member selected from the group consisting of an aliphatic polyester such as poly(lactic acid), a copolymer of lactic acid and glycolic acid, polycaprolactone, a copolymer of lactide and 1,4-dioxane-2-one, a copolymer of caprolactone and lactic acid, and a copolymer of caprolactone and glycolic acid, a polyorthoester, polyanhydride, polyphosphoamide, poly(amino acid), polyurethane, and di-, tri-, or multiblock copolymers of these hydrophobic polymers and hydrophilic poly(ethylene glycol). More preferably, it is a member selected from the group consisting of poly(lactic acid), copolymer of lactic acid and glycolic acid, polycaprolactone, a copolymer of lactide and 1,4-dioxane-2-one, and di-, tri-, or multiblock copolymers of these hydrophobic polymers and hydrophilic poly(ethylene glycol). The biodegradable polymers are biocompatible and their molecular weights are preferably within the range of 1,000˜100,000 daltons, and more preferably within the range of 2,000˜50,000 daltons.

The physiologically or biologically active agents can include peptide or protein drugs which require sustained physiological activity over an extend period of time, antiphlogistics, anti-cancer agents, antiviral agents, sex hormones, antibiotics, or anti-fungal agents. In detail, the physiologically active agents of the present invention include but are not limited to: peptide or protein drugs such as animal growth hormones including bovine growth hormone, porcine growth hormone, or sheep growth hormone, human growth hormone, granulocyte-colony stimulating factor (G-CSF), epithelial growth factor, bone morphogenic protein, erythropoietin, interferon, follicle stimulating hormone, leutenizing hormone, goserelin acetate, leuprorelin acetate, and leutenizing hormone-releasing hormone agonist including decapeptyl; antiphlogistics such as indomethacin, ibuprofen, ketoprofen, piroxicam, flubiprofen, and diclofenac; anti-cancer agents such as paclitaxel, doxorubicin, carboplatin, camptothecin, 5-fluorouracil, cisplatin, cytosine arabinose, and methotrexate; antiviral agents such as acyclovir and ribavirin; sex hormones such as testosterone, estrogen, progesterone, and estradiol; antibiotics such as tetracycline, minocycline, doxycycline, ofloxacin, levofloxacin, ciprofloxacin, clarithromycin, erythromycin, cefaclor, cefotaxime, imipenem, penicillin, gentamycin, streptomycin, and bancomycin; and anti-fungal agents such as ketoconazole, itraconazole, fluconazole, amphotericin-B, and griseofulvin.

The water-miscible organic solvents are non-toxic to the body. Typical examples of organic solvents are members selected from the group consisting of acetic acid, lactic acid, formic acid, acetone, acetonitrile, dimethyl formamide, dimethyl acetamide, dimethyl sulfoxide, dioxane, and N-methyl pyrrolidone and mixtures thereof. Preferably, the water-miscible organic solvent is a member selected from the group consisting of acetic acid, lactic acid, N-methyl pyrrolidone, or a mixture thereof. The water-miscible organic solvent may be used alone or in a mixture with water.

An O/O emulsion is formed by emulsifying the above-prepared polymer solution containing a physiologically active agent into water-miscible alcohol. The water-miscible alcohol used as a suspending medium in the present invention is miscible with the above organic solvent as well as with water. Typical examples of the alcohol are members selected from the group consisting of methanol, ethanol, propanol, isopropanol, ethylene glycol, diethylene glycol, triethylene glycol, propylene glycol, glycerine, and mixtures thereof. Preferably, the water-miscible alcohol of the present invention is a member selected from the group consisting of methanol, ethanol, isopropanol, propylene glycol, and mixtures thereof.

When the polymer solution containing the catalyst is directly emulsified into the water-miscible alcohol, a stable O/O emulsion may not be formed and the polymers precipitate in the medium since the water-miscible organic solvent dissolves rapidly in the water-miscible alcohol and, as a result, the solubility of the polymer decreases, rapidly and the polymers conglomerate together. In order to avoid this polymer precipitation, an emulsion stabilizer is preferably used in the present invention. When an emulsion stabilizer dissolves in the water-miscible alcohol(suspending medium), the viscosity of the medium increases and a stable O/O emulsion having no polymer precipitates can be formed.

The emulsion stabilizer of the present invention is preferably soluble in water and alcohol, is capable of increasing viscosity of the suspending medium (water-miscible alcohol) when dissolved in the medium, is non-toxic to the body and causes no environmental problems. Typical examples of emulsion stabilizers are: water-soluble synthetic polymers such as polyvinylpyrrolidone, poly(ethylene glycol), and poloxamer; cellulose derivatives such as hydroxypropyl cellulose and hydroxypropylmethyl cellulose, and preferably, polyvinylpyrrolidone and hydroxypropyl cellulose. The content of emulsion stabilizer in the water-miscible alcohol is preferably within the range of 0.1˜50% (w/v), and more preferably within the range of 0.2˜20% (w/v). The content of emulsion stabilizer can varied according to the-viscosity of the water-miscible alcohol needed. Nonionic surfactants such as polyoxyethylenesorbitan esters or sorbitan esters, which are generally used as emulsion stabilizers, can't provide the desirable effects in the solvent system of the present invention.

The water-miscible alcohol, wherein the emulsion stabilizer is dissolved, is stirred at a temperature of 10˜80° C., preferably 20˜60° C., at a speed of 200˜20,000 rpm. The polymer solution containing a physiologically active agent is then slowly added to the water-miscible alcohol wherein the emulsion stabilizer is dissolved, and the mixture is stirred for 5˜60 minutes to give a stable O/O emulsion. This emulsion is then added to a neutral or alkaline aqueous solution of pH 6˜12 at a temperature of 0˜30° C. to extract the organic solvent and alcohol, and to precipitate the microspheres with the catalyst from the solution. The resulting microspheres are then filtered and freeze-dried for use.

The neutral or alkaline aqueous solution is preferably a common buffer solution of pH 6˜10, and an alkaline aqueous solution is preferred when an organic acid, such as acetic acid, is used as the organic solvent.

The microspheres prepared by the method of the present invention can be used as an HA delivery carrier capable of localizing and providing for the sustained release of catalysts to a specific site after the HA is administered by subcutaneous, intramuscular, or intravenous injection to an animal or a human body.

The microspheres can also be used as a drug delivery carrier that, when injected directly into the disease site, releases a drug with a sustained rate and then degrades into small molecules that can be eliminated from the body.

Referring now to FIG. 2, there is shown an exploded view in perspective of a static mixing device for forming cross-linked HA as it is injected into the patient. Syringe 1 has two or three parallel internal chambers, each of which is intended to be filled with a cross-linked material such as DVS, a filler material such as hyaluronic acid, and a catalyst such as sodium bicarbonate solute. The chambers in syringe 1 are separated by barrier 4. When a pair of plungers 6 are forced into the chambers in syringe 1, the contents of the syringe exit via outlet 2 through outlet passages 3 and 5, flow through static mixing element 7 and exit conduit 9, and are intimately mixed to form a homogeneous mass which will rapidly polymerize following expulsion from outlet 11 of exit conduit 9. Static mixing element 7 is prevented from being expelled during use from the outlet end of exit conduit 9 by a suitable constriction in the inside diameter of exit conduit 9 proximate its outlet end.

In one embodiment, maximum efficiency of mixing is obtained by insuring that the inlet end 12 of the first mixing blade 13 of static mixing element 7 is generally perpendicular to the plain of contiguity between the two resin streams exiting syringe 1 through exit passages 3 and 5. Such perpendicular orientation is obtained using a locating tang in exit conduit 9, which locating tang serves to orient static mixing element 7 with respect to syringe 1.

Rotational alignment of exit conduit 9 with respect to syringe 1 is obtained using a suitable mounting means (e.g., a bayonet mount). Bayonet locking tabs 14 have locking prongs 15 and stop surfaces 17. Exit conduit 9 has locking ramps 19 and stop surfaces 21. Exit conduit 9 is mounted on syringe 1 by centering the inlet of exit conduit 9 over outlet 2 of syringe 1, while aligning exit conduit 9 so that it can be pushed between bayonet locking tabs 14. Exit conduit 9 is then inserted firmly over outlet 2, and rotated approximately 90° clockwise (as viewed from the exit end of the conduit) so that locking ramps 19 are wedged between locking prongs 15 and the main body of syringe 1, and stop surfaces 17 engage stop surfaces 21.

When so mounted, exit conduit 9 is fixably rotationally aligned with respect to syringe 1. In addition, through locating means described in more detail below, static mixing element 7 is fixably rotationally aligned with respect to exit conduit 7 and syringe 1. Static mixing element 7 and exit conduit 9 are firmly attached to syringe 1, but can be readily removed and discarded after use by rotating exit conduit 9 approximately 90° counterclockwise (as viewed from the exit end of the conduit) and pulling exit conduit 9 away from syringe 1.

Syringe 1, exit nozzle 2, exit passages 3 and 5, barrier 4, plungers 6, static mixing element 7, exit conduit 9, inlet edge 12, first mixing blade 13, bayonet locking tabs 14, and locking prongs 15 are as in FIG. 1. Static mixing element 7 is rotationally aligned within exit conduit 9 by one or more guides proximate the outlet end of exit conduit 9. Guides 24 and 25 are small inward projections in the bore of exit conduit 9, and have a “fish mouth” appearance when viewed in perspective. When viewed in isolation, locking guides 24 and 25 each resemble the nib of a fountain pen.

When static mixing element 7 is inserted into the inlet end of exit conduit 9, and pushed toward the outlet end of exit conduit 9, guides 24 and 25 serve to rotationally align static mixing element 7 within exit conduit 9. When leading edge 26 of the final mixing blade 28 of static mixing element 7 approaches the outlet end of exit conduit 9, guides 24 and 25 cause static mixing element 7 to rotate about its long axis until leading edge 26 abuts edge surface 24a of guide 24 or edge surface 25a of guide 25.

When a static mixing element is inserted sufficiently far into exit conduit 9 to strike cusp 33, the leading edge of the static mixing element is deflected by cusp 33 toward edge surface 24a or toward edge surface 24b, thereby providing the desired rotational alignment. Depending upon whether the static mixing element abuts against edge surface 24a or 24b of guide 24 (and against corresponding edge surface 25b or 25a of guide 25), the final orientation of the static mixing element will be in one of two positions, each of those positions being 180° of rotation apart from the other. Each position is equally acceptable as a means for optimizing the efficiency of the first blade of the static mixing element, since in either position the first mixing element will intersect the incoming streams of resin at an approximate right angle to the plane of contiguity between the incoming streams and subdivide the incoming streams equally.

Upon injection into the patient's body, the contents of the chambers are mixed: a first chamber containing a cross-linking material such as DVS, a second chamber containing hyaluronic acid (HA), and a third chamber containing a catalyst such as sodium bicarbonate solution.

The encapsulated temperature release chemicals and methods of this invention are useful in a variety of applications. The term “controlled temperature release” is used herein to mean that a chemical encapsulated in accordance with the preferred embodiment will release at a known rate into the HA in which it is mixed at a selected temperature to cause cross-linking of the HA. While any of a great variety of chemicals can be encapsulated in accordance with this invention and used in a variety of applications, the encapsulated chemicals and methods are particularly suitable for use in cosmetic applications. Further, the encapsulated chemicals and methods of this system are particularly suitable for encapsulating hydrogels, but they also provide excellent encapsulation and temperature release for dry particulate solid chemicals.

It will be understood by the artisan that the bioabsorbable HA delivery devices discussed herein may be formed out of hydrogels and/or polymer blends of glycolide and/or lactide homopolymer, copolymer and/or glycolide/lactide copolymer and polycaprolactone copolymers and/or copolymers of glycolide, lactide, poly (L-lactide-co-DL-lactide), caprolactone, polyorthoesters, polydioxanone, trimethylene carbonate and/or polyethylene oxide or any other bioabsorbable material. Similarly, it will be further understood that therapeutic agents suitable for timed release by the various embodiments of the HA delivery device described herein include antibiotic compositions, analgesics, lactoferrin and any other compositions effective for reducing infection and/or promoting healing of a wound formed at a surgical site. The therapeutic agents can include temperature release or otherwise controllable properties which can be provided by the hydrogels discussed above and/or the synthetic molecular level devices referenced above or other timed release agents or mechanisms known in the art. When synthetic molecular level devices are employed, they can be turned on and off or opened and closed by various stimuli such as sound or a magnetic field or other means. Therapeutic agents and/or delivery systems employing nanotechnologies can also be employed and these can include sustained release systems and other drug delivery systems known in the art, solubility enhancement, adjuvant carriers, manufactured neurons to aid in reversal of paralysis, nano-sized therapeutic agents and the like.

The release of the catalyst from the composition may be varied or controlled, for example, by the solubility of a biologically-active agent in aqueous tissue fluids, the distribution of the bioactive agent within the matrix, the size, shape, porosity, solubility and biodegradability of the composition, the type and amount of crystallization-controlling agent and/or an additive, triggering a synthetic molecular level device and/or the like. The relative amounts of bioabsorbable/biodegradable polymer and/or hydrogel in the HA delivery device in accordance with all of the embodiments of the present invention may vary widely, depending on the rate of dissolution of the polymer and/or hydrogel (and, therefore, the rate of catalyst release) desired. The polymer and/or hydrogel composition includes the therapeutic agent in an amount effective to provide the desired level of biological, physiological, pharmacological and/or therapeutic effect in the patient. There is generally no critical upper limit on the amount of the therapeutic agent included in the composition. The only limitation is a physical limitation for advantageous application (i.e., the therapeutic agent should not be present in such a high concentration that the consistency and handling of the composition is adversely affected). The lower limit of the amount of therapeutic agent incorporated into the composition will depend on the activity of the therapeutic agent and the period of time desired for treatment.

A variety of antibiotic drugs can be used in the implants to treat or prevent infection. Suitable antibiotics include many classes, such as aminoglycosides, penicillins, semi-synthetic penicillins, cephalosporins, doxycycline, gentamicin, bacitracin, vancomycin, methicillin, cefazolin and quinolines. Clindamycin has been reported to release readily from composites comprising polylactic acid. Anti-inflammatory agents such as hydrocortisone, prednisone, and the like may comprise the therapeutic agent. Substances useful for promoting growth and survival of cells and tissues or augmenting the functioning of cells, as for example, a nerve growth promoting substance such as a ganglioside, a nerve growth factor; a hard tissue growth promoting agent such as an osteoinductive growth factor, are also possible therapeutic agents suitable for incorporation within a modular drug delivery device of the present invention. The protein lactoferrin, an iron scavenger, has recently been shown to prevent the buildup of “biofilms” comprising bacterial colonies. The incorporation of lactoferrin into an implantable modular drug delivery system may be useful for preventing the formation of harmful biofilms at a surgical site.

The rate of release of a therapeutic agent from the modular drug delivery device generally depends on the concentration of the therapeutic agent in the composition and the choice of bioabsorbable polymer and/or hydrogel. For a particular polymer and/or hydrogel, the rate of release may further be controlled by the inclusion of one or more additives that function as a release rate modification agent, and by varying the concentration of that additive. The release rate modification additive may be, for example, an organic substance which is water-soluble or water insoluble. Useful release rate modification agents include, for example, fatty acids, triglycerides, other like hydrophobic compounds, organic solvents, plasticizing compounds and synthetic molecular level devices.

The inner content 6 of the implant is a composition that is composed mainly of hyaluronic acid. The term “hyaluronic acid” is used in literature to mean acidic polysaccharides with different molecular weights constituted by residues of D-glucuronic and N-acetyl-D-glucosamine acids, which occur naturally in cell surfaces, in the basic extracellular substances of the connective tissue of vertebrates, in the synovial fluid of the joints, in the endobulbar fluid of the eye, in human umbilical cord tissue and in cocks' combs. The term “hyaluronic acid” is in fact usually used as meaning a whole series of polysaccharides with alternating residues of D-glucuronic and N-acetyl-D-glucosamine acids with varying molecular weights or even the degraded fractions of the same, and it would therefore seem more correct to use the plural term of “hyaluronic acids”. The singular term will, however, be used all the same in this description; in addition, the abbreviation “HA” will frequently be used in place of this collective term. HA can also be defined as an unsulphated glycosaminoglycan composed of repeating disaccharide units of N-acetylglucosamine (GlcNAc) and glucuronic acid (GlcUA) linked together by alternating beta-1,4 and beta-1,3 glycosidic bonds. Hyaluronic acid is also known as hyaluronan, hyaluronate, or HA. The terms hyaluronan and hyaluronic acid are used interchangeably herein. More details on how to make the HA are discussed in commonly owned, co-pending application Ser. No. 13/353,316, filed Jan. 18, 2012, and entitled “INJECTABLE FILLER,” the content of which is incorporated by reference.

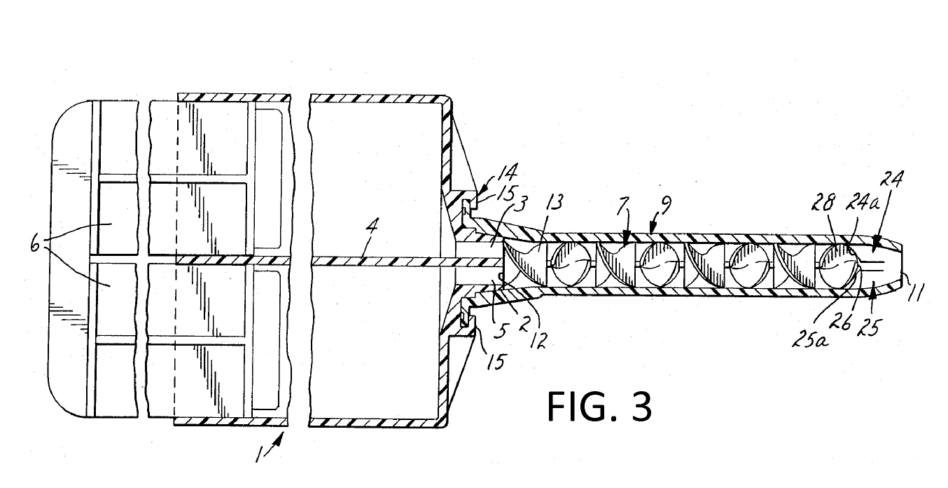

The injectors of FIG. 1 or FIGS. 2-3 can be used to inject HA for dermal filling or for filling a breast or butt or body implant, as shown in FIG. 4.

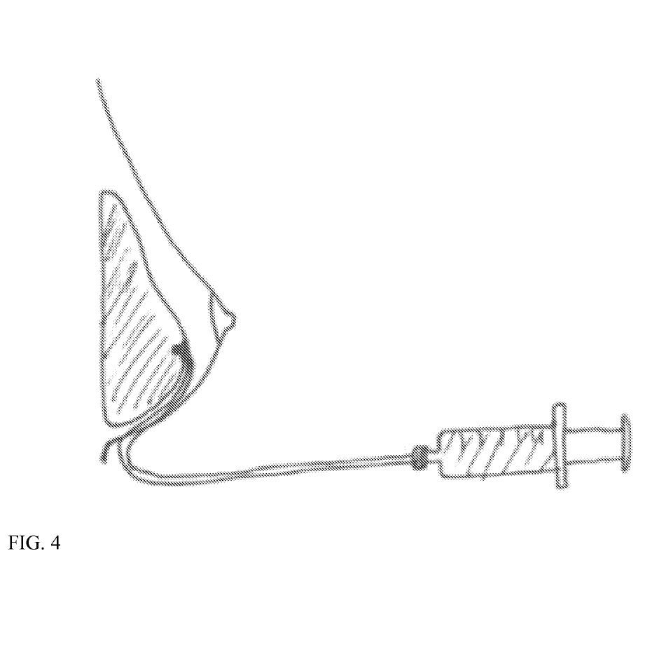

FIG. 5 shows an exemplary process to inject and cross-link materials at the same time. The process includes forming a biocompatible cross-linked polymer having a multi-phase mixture with a time release catalyst (702). Next, the process injects the mixture into a patient as a viscous fluid (704). After injection, the catalyst is activated to cross-link the polymer at a predetermined temperature after injection into a patient (706). Finally, the cross-linked polymers are used for augmenting soft tissue with the biocompatible cross-linked polymer (708).

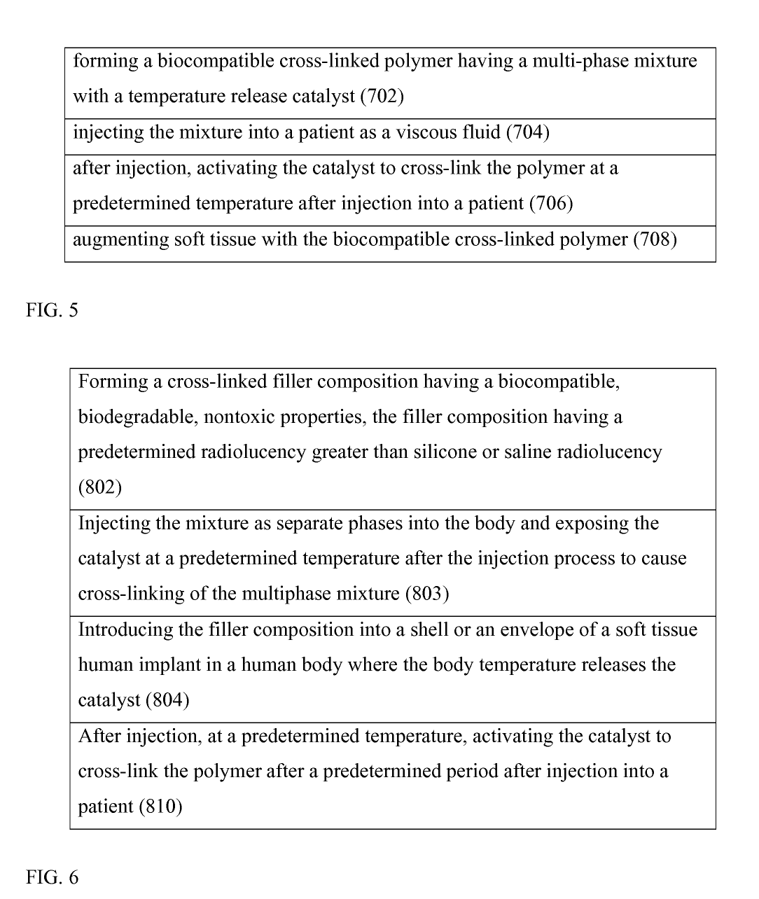

FIG. 6 shows another exemplary process to inject and cross-link materials at the same time. The process includes forming a cross-linked filler composition having a biocompatible, biodegradable, nontoxic properties, the filler composition having a predetermined radiolucency greater than silicone or saline radiolucency (802). The process also injects the mixture as separate phases into the body and mixing the mixture during the injection process to cause cross-linking of the multiphase mixture (803). The filler composition with HA, temperature sensitive catalyst, and cross linking materials is introduced into a shell or an envelope of a soft tissue human implant. After implantation of the shell or envelope into a lumen in a human body (804), at a predetermined body temperature, the catalyst is activated to cause cross-linking the filler composition. The cross linking reaction can occur outside the shell/envelope or in-situ inside the shell/envelope (810)

With certain HAs, the cross linking of the HA external to the shell can cause the cross-linked gel to become hardened and thus the HA may not be inserted into the shell easily with desired properties. A reversible cross-linking system can be used in one embodiment, where the cross links will be labile at extreme pH values, and at physiological pH, the cross-links become fixed. Two product streams can enter the shell, one is the product at an altered pH state and the other is the PBS, the neutralizer.

Gelling by either bioresponsive self-assembly or mixing of binary crosslinking systems, these technologies are useful in minimally invasive applications as well as drug delivery systems in which the sol-to-gel transition aids the formulation's performance. Moreover, not only does the chemical nature of the crosslinking moieties allow these systems to perform in situ, but they contribute dramatically to the mechanical properties of the hydrogel networks. For example, reversible crosslinks with finite lifetimes generate dynamic viscoelastic gels with time-dependent properties, whereas irreversible crosslinks form highly elastic networks.

The intrinsic properties of in situ forming gels add a new dimension of flexibility to large space augmentation such as that of the breast or butt or bodyparts. While the silicone filled shell gives the feel and touch of native tissue, the long term health and legal complications associated of foreign body reaction and biocompatibility cannot be avoided. The over the lifetime of the implant, the fact that silicone fluid finding its way to the tissue on the outside of the shell is a kinetic eventuality. Saline filled shell has been a reluctant alternative because its feel and aesthetic affect are far from natural. The best of both worlds alternative might be found in a native material such as hyaluronic acids. The required properties might be best satisfied in an in situ crosslinked hyaluronic acid, or ex situ crosslinked hyaluronic acid or super high molecular weight linear (uncrosslinked) hyaluronic acids.

The following are examples of in situ crosslinking method for hyaluronic acids:

1. Hyaluronic acids, hydrazide and aldehyde:

Doubly crosslinked networks composed of HA microgels and crosslinked hydrogels with tunable is coelasticity in the relevant frequency range have been proposed for vocal fold healing. These partially monolithic and partially living materials feature divinylsulfone-crosslinked HA particles that have been oxidized with periodate to produce surface aldehyde functionalities.

A derivative of hyaluronic acid (HA), comprising the steps of:

1.1. forming an activated ester at a carboxylate of a glucuronic acid moiety of hyaluronic acid;

1.2. substituting at the carbonyl carbon of the activated ester formed in step 1.1

1.3. a side chain comprising a nucleophilic portion and a functional group portion; and

1.4. forming a cross-linked hydrogel from the functional group portion of the hyaluronic acid derivative in solution under physiological conditions wherein the forming of a cross-linked hydrogel is not by photo-cross-linking.

2. Hyaluronic acid, dextran by forming a hydrazine

3. Functionalization of hyaluronic acid (HA) with chemoselective groups enables in situ formation of HA-based materials in minimally invasive injectable manner. One embodiment of HA modification with such groups primarily rely on the use of a large excess of a reagent to introduce a unique reactive handle into HA and, therefore, are difficult to control. FIG. 9 shows another embodiment with a protective group strategy based on initial mild cleavage of a disulfide bond followed by elimination of the generated 2-thioethoxycarbonyl moiety ultimately affording free amine-type functionality, such as hydrazide, aminooxy, and carbazate. Specifically, new modifying homobifunctional reagents may be synthesized that contain a new divalent disulfide-based protecting group. Amidation of HA with these reagents gives rise to either one-end coupling product or to intra/intermolecular cross-linking of the HA chains. However, after subsequent treatment of the amidation reaction mixture with dithiothreitol (DTT), these cross-linkages are cleaved, ultimately exposing free amine-type groups. The same methodology was applied to graft serine residues to the HA backbone, which were subsequently oxidized into aldehyde groups. The strategy therefore encompasses a new approach for mild and highly controlled functionalization of HA with both nucleophilic and electrophilic chemoselective functionalities with the emphasis for the subsequent conjugation and in situ cross-linking. A series of new hydrogel materials were prepared by mixing the new HA-aldehyde derivative with different HA-nucleophile counterparts. Rheological properties of the formed hydrogels were determined and related to the structural characteristics of the gel networks. Human dermal fibroblasts remained viable while cultured with the hydrogels for 3 days, with no sign of cytotoxicity, suggesting that the gels described in this study are candidates for use as growth factors delivery vehicles for tissue engineering applications.

4. The gelation is attributed to the Schiff base reaction between amino and aldehyde groups of polysaccharide derivatives. In the current work, N-succinyl-chitosan (S-CS) and aldehyde hyaluronic acid (A-HA) were synthesized for preparation of the composite hydrogels.

5. Injectable hyaluronic acid (HA) hydrogels cross-linked via disulfide bond are synthesized using a thiol-disulfide exchange reaction. The production of small-molecule reaction product, pyridine-2-thione, allows the hydrogel formation process to be monitored quantitatively in real-time by UV spectroscopy. Rheological tests show that the hydrogels formed within minutes at 37° C. Mechanical properties and equilibrium swelling degree of the hydrogels can be controlled by varying the ratio of HA pyridyl disulfide and macro-cross-linker PEG-dithiol. Degradation of the hydrogels was achieved both enzymatically and chemically by disulfide reduction with distinctly different kinetics and profiles. In the presence of hyaluronidase, hydrogel mass loss over time was linear and the degradation was faster at higher enzyme concentrations, suggesting surface-limited degradation.

Other Examples include:

A. To produce a crosslinked hyaluronic acid filler composition by in-situ cross linking Using Divinyl Sulfone to fill a 200 mL silicone shell:

| 1. Hyaluronic Acid (2M Dalton) | 1.5 | g |
|---|---|---|
| 2. NaOH (0.2N) | 50 | mL |
| 3. Combine the two and mix until completely dissolved | | |
| 4. Inject this hyaluronic acid/NaOH mixture into the silicone | 35 | μL |
| shell | | |
| 5. Divinyl sulfone | 150 | μL |
| 6. PBS (phosphate buffered saline) | 150 | mL |
| 7. Thoroughly mix the PBS and DVS | | |
| 8. Inject the PBS/DVS into the shell | | |

9. Mix vigarously together for homogeneous crosslinking reaction
10. Neutralize using an appropriate amount of acid such as hydrochloric
acid with the pH be monitored.
10. Use appropriately

B. To produce a crosslinked hyaluronic acid filler composition by in-situ cross linking Using 1,4-butane diol diglycidyl ether (BDDE) to fill a 200 mL silicone shell:

| | | |
|---|---|---|
| 1. Hyaluronic Acid (2M Dalton) | 2.0 | g |
| 2. NaOH (0.2N) | 50 | mL |
| 3. Combine the two and mix until completely dissolved | | |
| 4. Inject this hyaluronic acid/NaOH mixture into the silicone | 40 | μL |
| shell | | |
| 5. 1,4-butane diol diglycidyl ether | 150 | μL |
| 6. PBS (phosphate buffered saline) | 150 | mL |
| 7. Thoroughly mix the PBS and BDDE | | |
| 8. Inject the PBS/BDDE into the shell | | |

9. Mix vigarously together for homogeneous crosslinking reaction
10. Neutralize using an appropriate amount of acid such as hydrochloric
acid with the pH be monitored.
11. Use appropriately

The viscosity of these polymers could be controlled by using its pH properties. The low viscosity region during low pH environment helps with deployment of the augmentation gel because the gel has to be delivered through a small diameter tubing. Polymers that are pH sensitive are also called polyelectrolytes. The swelling properties of polyelectrolyte networks, which can be described in terms of the swelling rate and maximum solution uptake at equilibrium, depend on the physicochemical properties of the polymers and on the composition of the surrounding medium. Polyelectrolyte gels change their conformation with the degree of dissociation which is the function of quantities such as pH value, polarity of the solvent, ionic strength and temperature of the external environment solution.

### EXAMPLE C

Synthesis of biocompatible and biodegradable polyelectrolyte hydrogels based on polyvinyl pyrrolidone (PVP), gelatin and hyaluronic acid (HA) using gamma irradiation polymerization technique. The example polymers of C1 and C2 at pH 5 exponentially increased their water absorption properties. The addition of PVP and gelatin were for in-vitro handling and processing ease.

C1.

| | |
|---|---|
| PVP | 5 g |
| Hyaluronic acid | 1 g |

Mix well and expose the mixture to 30 kGy radiation

C2.

| | |
|---|---|
| Gelatin | 10 g |
| Hyaluronic acid | 1 g |

Mix well and expose the mixture to 30 kGy radiation

### EXAMPLE D

Synthesis of hyaluronic acid and polyvinyl alcohol at various respective ratios in an interpenetrating networks. The polyvinyl alcohol included in the polymer system was for ease of in-vitro handling and processing. Glutaraldehyde and hydrochloric acid were catalysts for the PVA reaction. The 1-ethyl-(3-3-dimethylaminopropyl) carbodiimide hydrochloride was the catalyst for the hyaluronic acid reaction. The two materials independently crosslinked at their primary structure levels while their secondary structures intertwined to create interpenetrating polymer networks. Examples D1, D2 and D3, at pH 4 exponential changed their water absorption properties.

D1.

| | |
|---|---|
| Hyaluronic acid | 3 g |
| Polyvinyl alcohol | 1 g |

D2.

| | |
|---|---|
| Hyaluronic acid | 1 g |
| Polyvinyl alcohol | 1 g |

D3.

| | |
|---|---|
| Hyaluronic acid | 1 g |
| Polyvinyl alcohol | 3 g |

Another preferred embodiment is filling a silicone shell with cross-linked hyaluronic acid material. This method required a high sheer mixer. The HA is cross linked using available cross-linkers such as divinyl sulfone, 1,4-butane diol diglycidyl ether in the presence of 0.1M sodium hydroxide. When the crosslinking reaction has completed, the HA gel is washed repeatedly until the residual cross-linker was no longer detectable in the HA gel, At this point, the cross-linked gel is blended with 10% water in shear mode to create uniform and small particles. The blended cross-linked material reformulated with un-cross-linked materials HA for injectability and longevity.

The implants of the present invention further can be instilled, before or after implantation, with indicated medicines and other chemical or diagnostic agents. Examples of such agents include, but are not limited to, antibiotics, chemotherapies, other cancer therapies, brachytherapeutic material for local radiation effect, x-ray opaque or metallic material for identification of the area, hemostatic material for control of bleeding, growth factor hormones, immune system factors, gene therapies, biochemical indicators or vectors, and other types of therapeutic or diagnostic materials which may enhance the treatment of the patient.

The present invention has been described particularly in connection with a breast or butt or body implant, but it will be obvious to those of skill in the art that the invention can have application to other parts of the body, such as the face, and generally to other soft tissue or bone. Accordingly, the invention is applicable to replacing missing or damaged soft tissue, structural tissue or bone, or for cosmetic tissue or bone replacement.

The following examples will enable those skilled in the art to more clearly understand how to practice the present invention. It is to be understood that while the invention has been described in conjunction with the preferred specific embodiments thereof, that which follows is intended to illustrate and not limit the scope of the invention. Other aspects of the invention will be apparent to those skilled in the art to which the invention pertains.

Although the present invention has been described in relation to particular embodiments thereof, many other variations and modifications and other uses will become apparent to those skilled in the art. It is preferred, therefore, that the present invention be limited not by the specific disclosure herein, but only by the appended claims. The other methods, used for characterization of the products according to one embodiment are described in the following examples which illustrate preferred embodiments of one embodiment without, however, being a limitation thereof. Variations and modifications can, of course, be made without departing from the spirit and scope of the invention.

What is claimed is:

1. A method for cosmetic augmentation, comprising:
forming a biocompatible cross-linked polymer having a multi-phase mixture with a temperature activated catalyst;
injecting the mixture into a patient as a homogeneous fluid;
activating the catalyst to cross-link the polymer at a predetermined temperature in a patient; and
augmenting soft tissue with the biocompatible cross-linked polymer.

2. The method of claim 1, comprising providing a predetermined controlled release of selected pharmaceutical substance to modulate soft tissue response to the polymer.

3. The method of claim 1, comprising cross-linking the polymer in a shell inside the patient.

4. The method of claim 1, wherein the polymer comprises one of:
collagens, hyaluronic acids, celluloses, proteins, saccharides.

5. The method of claim 1, wherein the polymer comprises an extracellular matrix of a biological system.

6. The method of claim 1, comprising using cross linkers and forming homo-polymers or to form copolymers by crosslinking with other polymer species.

7. The method of claim 1, comprising adding a substance to the composition for biocompatibility.

8. The method of claim 1, comprising controlling drug releases at predetermined timing in anticipation of an onset of a negative physiological event in response to invading foreign bodies.

9. The method of claim 1, comprising fast releasing the composition.

10. The method of claim 1, comprising adding anesthetics, lidocaine or compound to reduce or eliminate acute inflammatory reactions to the pharmaceutical substance.

11. The method of claim 1, comprising adding one or more compositions selected from the group consisting of steroids, corticosteroids, dexamethasone, triamcinolone.

12. The method of claim 1, comprising providing a slow release substance to the pharmaceutical substance.

13. The method of claim 1, comprising providing an antiproliferative compound.

14. The method of claim 1, wherein the substance comprises paclitaxel, serolimas.

15. The method of claim 1, comprising controlling the scar formation process around a foreign body including capsular formation.

16. The method of claim 1, comprising providing a medium release to the pharmaceutical substance.

17. The method of claim 1, comprising optimizing degradation profile of the composition.

18. The method of claim 1, comprising minimizing migration of the composition.

19. The method of claim 1, comprising controlling an average molecular weight (Mn) and the polydispersity index.

20. The method of claim 1, comprising characterizing a target tissue, and maintaining a consistency of the composition in particle size and population densities.

21. The method of claim 1, comprising co-cross-linking glycosaminoglycan chemically with at least one other polymer including hyaluronan or hylan.

22. The method of claim 1, comprising adding a biodegradable surfactant or plasticizer to reduce surface tension of material as it is injected into a patient through a small diameter needle.

23. The method of claim 1, comprising storing the catalyst as microspheres and melting the microspheres at body temperature to release the catalyst.

24. The method of claim 1, comprising modeling a 3D model of a human body and continuously updating a current shape of breast or butt from the 3D model to fit to a desired shape.

25. The method of claim 1, comprising injecting with a mechanical pump the biocompatible crosslinked polymer under soft tissue in a minimally invasive manner.

26. A method for cosmetic augmentation, comprising:
forming a biocompatible cross-linked polymer having a multi-phase mixture with a predetermined controlled release of selected pharmaceutical substance to modulate soft tissue response to the polymer;
injecting the mixture into a patient and releasing a catalyst at a predetermined body temperature to cross-link the polymer in the patient;
filling a semi-permeable shell with the pharmaceutical substance; and
augmenting soft tissue with the biocompatible cross-linked polymer.
